(19) United States
(12) Patent Application Publication
(10) Pub. No.: US 2024/0238493 A1
(43) Pub. Date: Jul. 18, 2024

(54) DIALYSIS SYSTEM INCORPORATING A TOXIN-REMOVAL LOOP

(71) Applicant: University of Washington, Seattle, WA (US)

(72) Inventors: Guozheng SHAO, Seattle, WA (US); Bruce J. Hinds, Seattle, WA (US)

(73) Assignee: University of Washington, Seattle, WA (US)

(21) Appl. No.: 18/554,284

(22) PCT Filed: Apr. 4, 2022
(86) PCT No.: PCT/US2022/023305

§ 371 Date: Oct. 6, 2023

Related U.S. Application Data
(60) Provisional application No. 63/171,503, filed on Apr. 6, 2021.

(51) Int. Cl. A61M 1/16; A61M 1/28; A61M 1/36; B01D 61/00; B01D 61/02; B01D 69/02; B01D 69/08

(52) U.S. Cl. CPC A61M 1/1621; A61M 1/281; A61M 1/3681; B01D 61/002; B01D 61/025; B01D 69/02; B01D 69/08; A61M 2202/049; A61M 2209/088

(57) ABSTRACT

A protected fluid circuit can utilize osmotic membranes or other membrane types that are highly selective to urea transport. The membranes can achieve sufficient diffusional flux of urea in a forward osmosis geometry. When combined with a oxidation unit, this protected geometry can have a high selective flux of urea from the spent dialysate side to the urea removal side; balance of fluid (water) levels in patients by forward osmotic flow by controlling water evaporation rate through an vapor permeable membrane; protection of patient dialysate/blood loop from oxidation by-products; and an ability to optimize oxidation system performance (pH, ionic strength, other electrolytes, etc.) that would be otherwise incompatible with blood contact.

### CROSS-REFERENCE(S) TO RELATED APPLICATION(S)

This application claims the benefit of U.S. Provisional Application No. 63/171,503, filed on Apr. 6, 2021, the entire contents of which are herein expressly incorporated by reference.

### BACKGROUND

End-stage kidney disease (ESKD) is forcing nearly four million people worldwide to receive dialysis to sustain life, with this number projected to continue to grow. Dialysis treatments are a physical and economic burden to patients, their families and society.

Moreover, conventional hemodialysis is considered suboptimal in terms of rates of rehabilitation, quality of life, mortality and morbidity.

Extended and more frequent dialysis have shown potential improvements in health-related outcomes compared with conventional thrice-weekly in-center dialysis therapy.

However, a significant challenge to at home dialysis treatment is a continuous dialysis fluid supply that would enable portability. Presently, about 120 L (about 120 Kg) of fresh dialysate is used per session and is not possible to carry in a portable system. Portability may possibly be achieved by having a dialysis system where the spent dialysate is continuously being regenerated. Modes of urea removal that have been tried for regenerating dialysate include, but are not limited to, urease-based enzymes, sorbent-based, and oxidation-based elements.

Uremic toxins that are removed through dialysis include, but are not limited to urea, Na+, K+, Phenylacetic acid, Creatinine, Uric acid, Phenylacetylglutamine, Phosphorous, Hippuric acid, Oxalate, Dimethylamine, p-cresyl sulfate, 32-microglobulin, Phenyl sulfate, Indican, p-cresylglucuronide, 3-Hydroxyphenylacetic acid, Trimethylamine-N-oxide, 4-hydroxyhippuric acid, Indoxyl sulfate, 3-Hydroxyhippuric acid, Ca²⁺, Complement factor D, Acrolein, Guanidinosuccinic acid, Indole-3-acetic acid, Cystatin C, Methylguanidine, Symmetric dimethylarginine, and Guanidinoacetic acid. Of these, urea is the single most abundant small molecule metabolic waste that needs to be removed during dialysis, either in a single pass system or with regenerated dialysate.

With a combined mass of over 23.0 g, these uremic toxins interfere with the urea-removal elements of dialysis circuits, regardless of the type of urea-removal element chosen.

Also, large amounts of nutrients that coexist with urea in the spent dialysate will hamper urea removal efficiency if there is insufficient chemical selectivity in membranes, adsorbents, or conversion systems. The inevitable loss of nutrients during conventional single-pass HD are a side effect of uremic toxin removal and is an important component of dialysis related catabolism. The loss of nutrients include but are not limited to albumin, amino acids, globulins, glucose, cholesterol, bilirubin, and transferrin. These lost solutes are generally involved in anti-oxidant, anti-inflammatory, toxin removal, and vasodilating effects with their reduced concentration, contributing additional adverse effects to uremia treatment. If a dialysis regeneration system is developed that selectively does not remove amino acids glucose and functional proteins, then HD nutrition loss would be effectively eliminated.

Another important consideration is the purity of the dialysate water source. The large volume (about 120 L) of conventional dialysate imposes a significant hurdle for portability, especially with the stringent requirement on water quality followed by electrolyte balancing. This cost could be largely mitigated by switching to a dialysate regeneration system.

FIG. 1 shows the WAK dialysis circuit including a dialysate loop 104, separated from a patient blood circuit 102 by a dialysis membrane 110. The blood circuit 102 recirculates the patient's blood. The dialysate loop 104 recirculates a dialysate fluid. The toxins from the patient's blood are transferred to the dialysate through the membrane 110. The dialysate then travels through the urea removal cartridge 114. After the dialysate flows through the bottom four layers of the cartridge to remove urea/ammonia/salts, the top layer of activated carbon is largely sufficient to remove other uremic toxins before reuse through the membrane 110 once again. It is also important to note that activated carbon is largely inert to both urea and glucose, allowing glucose to stay in the dialysate loop.

Despite the remarkable innovations in the designs of devices and modes of operation, there are still challenges with urease, sorbents, and oxidation methods for regenerating dialysate.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one Embodiment, a liquid dialysis circuit comprises a dialysate loop 204, separated from a patient blood circuit 202 by a dialysis membrane 210, and a toxin-removal loop 206 separated by a toxin-selective membrane 212 configured to selectively pass the toxin from the dialysate loop 204 to the toxin-removal loop 206.

In one Embodiment, the toxin is a uremic toxin.

In one Embodiment, the toxin-selective membrane 212 is an osmotic membrane.

In one Embodiment, the toxin-selective membrane 212 is configured to operate as a forward-osmosis membrane.

In one Embodiment, the toxin-selective membrane 212 is configured to operate as a reverse-osmosis membrane.

In one Embodiment, the toxin-selective membrane 212 operates based on the mechanism of ion-exchange (to pursue negative-charged toxins), affinity binding, ultrafiltration, reverse osmosis or forward osmosis.

In one Embodiment, the toxin-removal loop 206 comprises non-biocompatible liquid.

In one Embodiment, the dialysate loop 204 comprises a liquid filter 214 configured to remove a uremic toxin from a dialysate liquid flowing through the dialysate loop 204.

In one Embodiment, the toxin-removal loop 206 comprises a liquid filter 216 configured to remove a uremic toxin or oxidation product from a toxin-removal liquid flowing through the toxin-removal loop 206.

In one Embodiment, the toxin-removal loop 206 comprises a toxin-removal element 218 in liquid contact with a toxin-removal liquid flowing through the toxin-removal loop 206.

In one Embodiment, the toxin-removal element 218 is selected from a photo-oxidation element, an electro-oxidation element, and combinations thereof.

In one Embodiment, the toxin-removal element 218 is selected from enzyme (urease)-based, bacteria-based (bioreactor), sorbent-based, oxidation-based, and combinations thereof.

In one Embodiment, the toxin-removal element 218 includes a photo-oxidation element.

In one Embodiment, the liquid dialysis circuit is capable of filtering at least 0.625 g/hour of toxin from the dialysate loop.

In one Embodiment, the liquid dialysis circuit is capable of removing 0.5 L-4.0 L of water from the dialysate loop 204 in 24 hours.

In one Embodiment, the liquid dialysis circuit is configured to controllably remove water from the dialysate loop 204 via a photo-oxidation element 218 in the toxin-removal loop 206.

In one Embodiment, the dialysate loop 204 is configured to contain a dialysate and for the dialysate to receive uremic blood toxins from the patient's blood across the dialysis membrane 210.

In one Embodiment, wherein the dialysate loop 204 is configured to received used peritoneal fluid.

In one Embodiment, the toxin-selective membrane includes hollow fiber membranes.

In one Embodiment, the toxin-selective membrane has a surface area for mass transfer of at least 2.4 m².

In one Embodiment, the toxin-removal loop flows inside of the hollow fiber membranes, and the dialysate loop flows on the outside of the hollow fiber membranes.

In one Embodiment, the flow of the toxin-removal loop is at least 36 mL/min.

In one Embodiment, the flow of the dialysate loop is at least 3.7 times the flow of the toxin removal loop.

In one Embodiment, a kidney dialysis system comprises the liquid dialysis circuit of any of the preceding Embodiments.

In one Embodiment, the kidney dialysis system is a hemodialysis system, a peritoneal dialysis system, a hemofiltration system, or a hemodiafiltration system.

In one Embodiment, the kidney dialysis system has a form factor selected from the group of portable, wearable, movable, and fixed.

In one Embodiment, the kidney dialysis system is configured for in-home or in-dialysis center use.

In one Embodiment, a method of treating a patient with toxified blood comprises:
a. flowing toxified liquid from the patient into a liquid dialysis circuit of any of the preceding Embodiments, wherein the toxified liquid flows along a dialysis membrane, allowing uremic toxins to pass into the dialysate loop to produce toxified dialysate;
b. in the dialysate loop, flowing the toxified dialysate along the toxin-selective membrane to produce detoxified dialysate in the dialysate loop and toxified fluid containing electrolytes in the toxin-removal loop, wherein the detoxified dialysate is returned towards the dialysis membrane; and
c. in the toxin-removal loop, flowing the toxified fluid containing electrolytes into a toxin-removal element, to produce detoxified fluid containing electrolytes, wherein the detoxified fluid containing electrolytes is returned towards the toxin-selective membrane.

In one Embodiment, the toxified liquid is blood or peritoneal dialysis fluid.

In one Embodiment, the method further comprises a step of adding electrolytes to the fluid containing electrolytes.

In one Embodiment, a method of regenerating a dialysate fluid, comprises:
a. flowing a toxified dialysate along a toxin-selective membrane to produce detoxified dialysate in the dialysate loop and toxified fluid containing electrolytes in a toxin-removal loop; and
b. in the toxin-removal loop, flowing the toxified fluid containing electrolytes into a toxin-removal element, to produce detoxified fluid containing electrolytes, wherein the detoxified fluid containing electrolytes is returned towards the toxin-selective membrane.

In one Embodiment, a liquid dialysis circuit, comprises:
a dialysate loop; and
a toxin-removal loop separated by a toxin-selective membrane configured to selectively pass a toxin from the dialysate loop to the toxin-removal loop.

In one Embodiment, the toxin-selective membrane includes hollow fiber membranes.

In one Embodiment, the toxin-selective membrane has a surface area for mass transfer of at least 2.4 m².

In one Embodiment, the toxin-removal loop flows inside of the hollow fiber membranes, and the dialysate loop flows on the outside of the hollow fiber membranes.

In one Embodiment, the flow of the toxin-removal loop is at least 36 mL/min.

In one Embodiment, the flow of the dialysate loop is at least 3.7 times the flow of the toxin removal loop.

In one Embodiment, the dialysate loop includes a uremic toxin.

In one aspect, a liquid dialysis circuit, is provided that includes a dialysate loop, separated from a patient blood circuit by a dialysis membrane, and a toxin-removal loop separated by a toxin-selective membrane configured to selectively pass the toxin from the dialysate loop to the toxin-removal loop.

In another aspect, a kidney dialysis system is provided that includes a liquid dialysis circuit as disclosed herein.

In yet another aspect, a method of treating a patient with toxified blood, is provided that includes:
a. flowing toxified liquid from the patient into a liquid dialysis circuit as disclosed herein, wherein the toxified liquid flows along a dialysis membrane, allowing uremic toxins to pass into the dialysate loop to produce toxified dialysate;
b. in the dialysate loop, flowing the toxified dialysate along the toxin-selective membrane to produce detoxified dialysate in the dialysate loop and toxified electrolyte in the toxin-removal loop, wherein the detoxified dialysate is returned towards the dialysis membrane; and
c. in the toxin-removal loop, flowing the toxified electrolyte into a toxin-removal element, to produce detoxified electrolyte, wherein the detoxified electrolyte is returned towards the toxin-selective membrane.

A portable dialysis device offers the potential for extended treatment and much smoother blood serum composition change over time that mimics the physiological condition. Quality of patient life will also dramatically improve compared to thrice weekly 4 h treatments. A challenge for a portable dialysis device is to regenerate a limited amount of dialysate, so that it can be continuously used for the further removal of blood toxins. Urea removal can be achieved with urease hydrolyzing urea into ammonium and bicarbonate, which have to be removed with additional ion exchange materials. Sorbents with tens of mg/g binding capacity towards urea could also be used to remove urea and simplify the electrolyte balancing process. Oxidation-based urea removal has the ability to run continuously.

Despite all their potential advantages in urea removal, the promise of urease, sorbent and oxidation cannot be fully optimized as long as urea and other blood serum molecules coexist in the same circuit. In particular, the presence of the plethora of small and medium MW molecules reduces efficiency of enzymes and adsorbents by competition. Existing membrane technology, such as RO membranes could be utilized to achieve the selective separation of urea into a urea-only capture loop.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic illustration of a conventional wearable artificial kidney dialysis system;

FIG. 2 is a schematic illustration of a dialysis system including a toxin-removal loop in accordance with one embodiment;

FIG. 3 is a schematic illustration of a device including hollow fiber membranes;

FIG. 4A is a schematic illustration of a urease-based toxin-removal element;

FIG. 4B is a schematic illustration of a sorbent-based toxin-removal element;

FIG. 4C is a schematic illustration of a photooxidation-based toxin-removal element;

FIG. 5 is chart showing the molecular weight cutoff of different membrane classes with examples of allowed species in each regime;

FIG. 6A is a schematic illustration of urea photooxidation by POUR (photooxidation urea removal) device using 10 mM urea dissolved in either 0.15 M NaCl or spent dialysate collected from clinic, used in the Examples;

FIG. 6B is a schematic illustration of a combined FO-POUR (forward osmosis photooxidation urea removal) setup, used in the Examples;

FIG. 7 is a graphical representation of urea removal rate in 24 h and corresponding mass transfer coefficient using a commercially available AQUAPORIN® FO cartridge at different inner fiber flow rates, and the outer flow rate of 10 mM urea dialysate feed is 3.7 times the inner fiber flow rate, from the Examples;

FIG. 8A is a bar graph representation of cytotoxicity measured by the MTT assay (untreated 3T3 cells and latex control samples with different dilution factors), from the Examples;

FIG. 8B is a bar graph representation of cytotoxicity measured by the MTT assay (untreated 3T3 cells and t=0 h samples from spent dialysate and POUR, as-is or treated with activated carbon), from the Examples; and

FIG. 8C is a bar graph representation of cytotoxicity measured by the MTT assay (untreated 3T3 cells and t=24 h samples collected from stand-alone spent dialysate separate from the FO-POUR device and collected from spent dialysate and POUR collected within the FO-POUR device), from the Examples.

### DETAILED DESCRIPTION

Portable and wearable hemodialysis has the potential to improve health outcomes and quality of life for patients with kidney failure at reduced costs. Urea removal, required for dialysate regeneration, is a central function of any existing/potential portable dialysis device. Urea in the spent dialysate coexists with non-urea uremic toxins, nutrients, and electrolytes, all of which will interfere with the urea removal efficiency, regardless of whether the underlying urea removal mechanism is based on urease conversion, direct urea adsorption or oxidation. This disclosure describes a toxin-removal loop separated from the dialysate loop by a toxin-selective membrane. The toxin-removal loop additionally includes at least one toxin-removal element, the performance of which can be improved and can advantageously enable regeneration of dialysate.

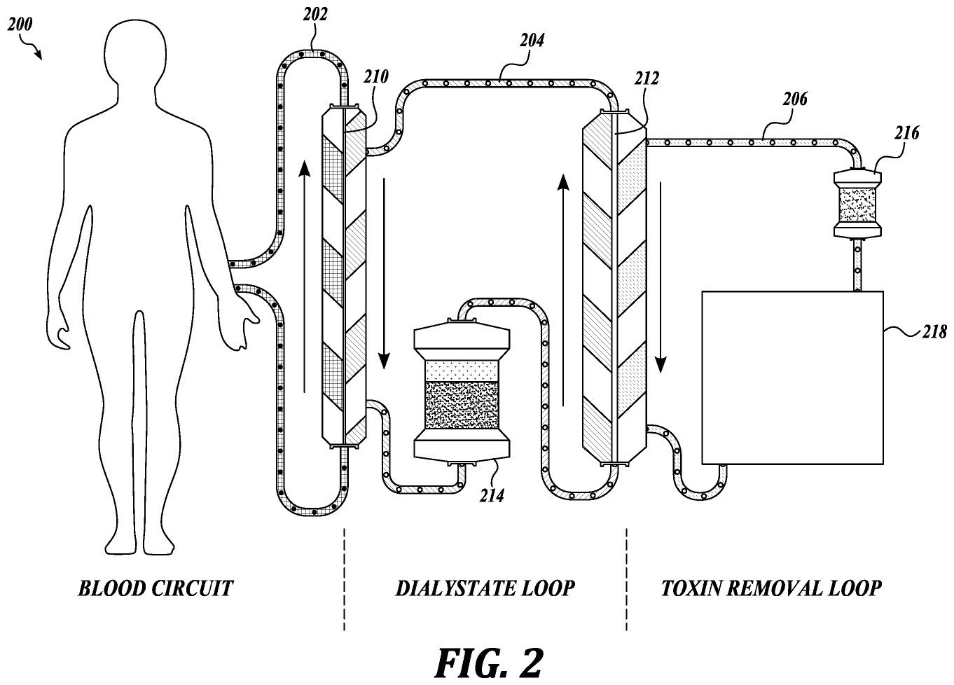

Referring to FIG. 2, one embodiment of a liquid dialysis circuit is illustrated, the liquid dialysis circuit includes a dialysate loop 204, separated from a patient blood circuit 202 by a dialysis membrane 210. The liquid dialysis circuit includes a toxin-removal loop 206 separated by a toxin-selective membrane 212 configured to selectively pass the toxin from the dialysate loop 204 to the toxin-removal loop 206. The liquid dialysis circuit includes a toxin-removal element 218. The dialysate in loop 204 can be any commercial or conventional dialysate currently used. The fluid in loop 206 can include, for example, saline solutions, aqueous solution mixed with non-NaCl electrolytes, aqueous solution of acidic or basic pH values. The toxin-removal loop 206 can protect the toxin-removal element 218 by using a toxin-selective membrane 212 in contact with the dialysate. For example, the urea-removal elements described herein can benefit from a urea-selective membrane 212 separating the dialysate loop 204 and the toxin-removal loop 206. In one embodiment, the toxin to be removed is a uremic toxin. In one embodiment, the toxin to be removed is urea.

As used herein, “loop” or “circuit” denote one or more conduits that carry fluid through one or more devices. The blood flow in the blood circuit 202 can be single pass, meaning fresh or new blood is continuously being fed into the blood circuit 202. The dialysate and fluid flows in loops 204 and 206 is designed to be multiple pass, meaning the fluid is being regenerated and can be used for extended periods. The flow in loops 204, 206 can flow in a continuous or semi-continuous manner so that the fluid circulates repetitively through the conduit and the various devices in the loops. The flow may be created by a pump, such as a peristaltic pump. Chemical species and/or fluids are allowed to be transferred between the dialysate loop 204 and the blood circuit 202 and between the dialysate loop 204 and the toxin-removal loop 206, through membranes 210 and 212, respectively. External fluids and/or nutrients may also be introduced into the loops 204, 206 and circuit 202, and fluids within the loops 204, 206 and circuit 202 may also be removed continuously or semi-continuously. In one embodiment, a loop may have a constant flow rate through the loop. In one embodiment, the flow of a loop may increase of decrease over time.

Using urea as an example, the toxin-removal loop 206 is separated by a urea-selective membrane 212 configured to selectively pass urea from the dialysate loop 206 to the toxin-removal loop 206. In this case, the dialysate loop 204 in FIG. 2 includes a cartridge 214, wherein the cartridge 214 includes a similar amount of activated carbon adsorbent (bottom layer) to adsorb non-urea toxins and potentially add other selective adsorbents (top layers) as needed or developed. Activated charcoal is a typical filter material, but any other compatible filter or method can also be used.

In the liquid dialysis circuit shown in FIG. 2, the urea from the dialysate loop 204 is removed across the urea-selective membrane 212 along with other unavoidable compounds. The urea is then removed from the urea loop 212 by any of the urea-removal elements. Here, the urea-removal element 219 can include a photooxidation urea removal element (POUR). When using a POUR as the urea-removal element 218, it is also advantageous to include an additional activated carbon filter 216 in the toxin-removal loop 206 to remove oxo-chloro species that can be a by-product during urea oxidation. Urea is used as a representative toxin, but it is understood that other uremic toxins can be removed from the dialysate loop 204 to the toxin-removal loop 206 by selection of the appropriate toxin-selective membrane 212.

Furthermore, more than one toxin-removal loops each with a different toxin-selective membrane can be included to remove more than one toxin. Alternatively, more than one different toxin-selective membrane may be used at the interface between the dialysate loop 204 and the toxin-removal loop 206. The toxin-removal element 218 can be based on enzyme (urease)-based, bacteria-based (bioreactor), sorbent-based, and oxidation-based. FIG. 2 shows an embodiment of a photooxidation urea-removal element.

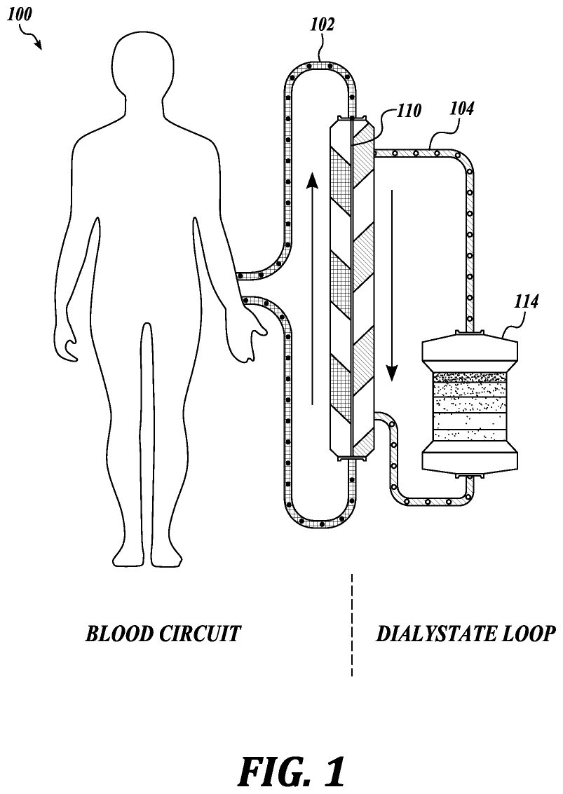

In this disclosure, the dialysis system of FIG. 2 may be compared to the conventional WAK system of FIG. 1. However, the use of a toxin-removal loop 206 and toxin-selective membrane 212 described with respect to FIG. 2 can be applied to other dialysis systems, such as a hemodialysis system, a peritoneal dialysis system, a hemofiltration system, or a hemodiafiltration system. Further, the kidney dialysis system of this disclosure can be configured to have a form factor selected from the group of portable, wearable, movable, and fixed systems, and the kidney dialysis system can be configured for in-home or in-dialysis center use.

### Urea-Removal Elements

The present disclosure aims to improve the operation of the currently available urea-removal elements by including an additional toxin-removal loop 206 separated from the dialysate loop 204 by a toxin-selective membrane 212. The toxin-selective membrane 212 can selectively pass urea, while blocking other compounds, into the toxin-removal loop 206 thereby improving the operation of the toxin-removal element 218.

In one embodiment, the toxin-removal element 218 can be selected from a photo-oxidation element or electro-oxidation element. In one embodiment, the toxin-removal element 218 can be selected from enzyme (urease)-based, bacteria-based (bioreactor), sorbent-based, oxidation-based, and combinations thereof. In one embodiment, the toxin-removal element 218 is a photo-oxidation element.

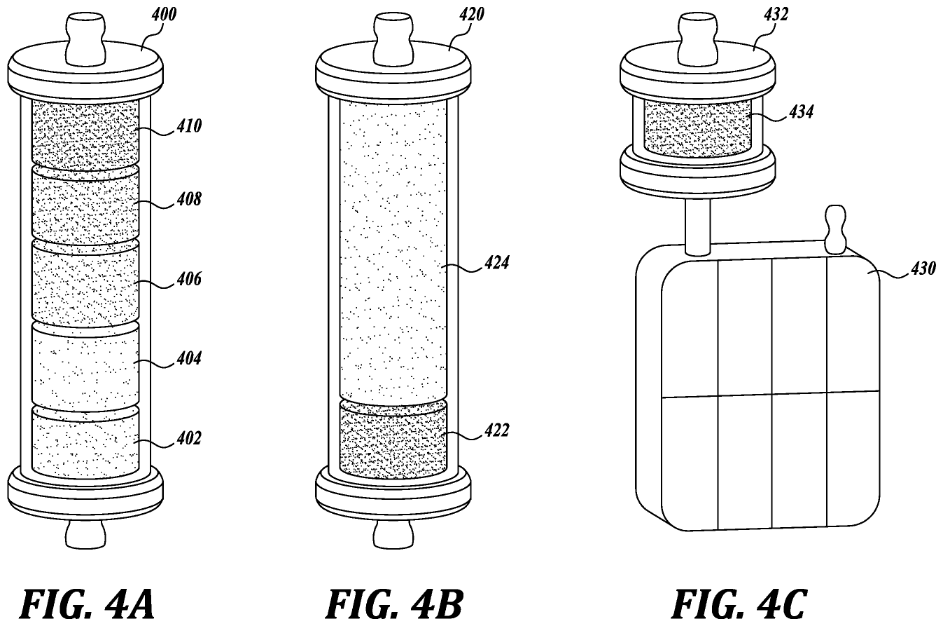

Urea removal has been removed by enzymes, adsorbents, anodic oxidation, bacterial degradation, polymer membrane capsules, electrodialysis, and reverse osmosis. The methods of urea removal, that have been developed in the context of a portable dialysis device that regenerates dialysate, can be divided into three categories: urease conversion followed by ion exchangers (FIG. 4A), direct adsorption (FIG. 4B), and oxidation (FIG. 4C).

### Urease Based Urea Removal

A dialysis treatment can be carried out with a dialysis machine using the urease enzyme as a urea removal method, such as the conventional REDY® (REgenerative DialYsis) system. The process starts with urease catalyzing the hydrolytic decomposition of urea and converting it into ammonium and bicarbonate, which are readily adsorbed by several cartridge elements. The capture of ammonium is beneficial, since it is toxic in the blood stream at low concentrations of about 100 μM. The urease is immobilized onto supports and is able to keep its activity as a robust enzyme. The immobilized urea has been found to be less sensitive to pH changes and maintains its activity for a longer time period. Immobilization of urease has been carried out in many different types of scaffolds. Chemical immobilization is generally preferred over physisorption due to the higher stability of fixation. However, as a result of the mild reaction conditions of urease fixation, even chemical bonding such as amine epoxy curing, N-(3-dimethylaminopropyl)-N′-ethylcarbodiimide hydrochloride (EDC), N-cyclohexyl-N′-(b-[N-methylmorpholino]ethyl)carbodiimide p-toluenesulfonate (CMC), and glutaraldehyde (GA) are reversible and subjected to attack from amine groups.

Referring to FIG. 4A, a urease removal element used as the toxin-removal element 218 in FIG. 2 can be provided as a cartridge 400. The cartridge 400 can include a first layer 402 functioning as a “scavenger layer” that will remove non-urea uremic toxins to some degree, but has a primary purpose to protect the urease from species that can deactivate or desorb it. Following the scavenger or first layer 402, the cartridge 400 includes a urease layer 404. Directly after the urease layer 404, the cartridge 400 can include a zirconium phosphate layer 406 which will use its pre-loaded Na⁺ and H⁺ to exchange for NH₄₊ produced by the urease layer 404, as well as the other cations including K⁺, Ca²⁺, and Mg². Zirconium phosphate has a high binding selectivity towards ammonium. Ammonium concentration could be reduced to less than 1 mg/L. After the zirconium phosphate layer 406, the cartridge 400 may include a zirconium oxide and zirconium carbonate layer 408 that can remove the excessive phosphate and other anions exchanged or leached from previous layers. A layer 410 of activated carbon can be placed before the exit for further trapping of non-urea uremic toxins and any desorbed urease. In particular, desorbed urease is hazardous since it can produce ammonia in the returned dialysate downstream of the zirconium phosphate and enter the blood stream.

### Sorbent Based Urea Removal

Referring to FIG. 4B, another embodiment of a toxin-removal element 218 is a high-capacity urea sorbent cartridge 420. The cartridge 420 can include a first layer of activated carbon 422 and a second layer of one or more sorbents 424. Sorbents remove urea based on the reaction with an electrophilic carbonyl group such as an aldehyde, or molecular size selection using materials such as activated carbon, zeolites and MXene. A wide range of urea binding capacity is possible, including up to 0.35 g/g. However, once applied in a medium close to the serum or spent dialysate, the urea binding capacities can drop to less than 1 mg/g. For example, activated carbon is effective at removing uremic toxins such as creatinine, uric acid, indicant, phenols, guanidines and organic acids. The pores of carbide-derived mesoporous carbon can be tailor-made with pores that fit exactly to proinflammatory compounds. With regard to urea, there is significant adsorption (>5 mg/g) at other than physiological serum conditions. The adsorption capacity of activated carbon around physiological urea (10 mM urea) concentration is about 1 mg/g.

A drop in urea binding capacity can occur in other sorbents when applied in the spent dialysate. Ti₃C₂Tₓ MXene can adsorb urea at a capacity of about 16 mg/g at 37° C. with equilibrium concentration of 25 mM. In contrast, when exposed to dialysate from patient, MXene exhibited only about 0.5 mg/g capacity under similar conditions, losing over 96% of its binding capacity. Similarly, oxidized starch was found to adsorb urea at a capacity of up to 8 mg/g under pH 7.4 at 37° C. through the aldehyde group, which will also adsorb other amine group-containing molecules. A polymeric sorbent based on phenylglyoxaldehyde (PGA) modification on surface polystyrene beads demonstrated over 36 mg/g urea binding capacity at 37° C. with 30 mM urea in phosphate-buffered saline. The binding mechanism of the PGA towards urea is based on its interaction with the amino group. Another sorbent can include a zeolite, which has an impressive urea binding capacity of about 30 mg/g at 37° C. with pH of 5.4 in solution with starting urea of 8.6 mM.

The sorbent-based urea removal cartridge 420 is simpler in construction compared to enzyme systems. A variety of high-capacity sorbents of urea have been developed that work under simple physiological conditions, i.e., at 37° C., about 0.15 M ionic strength and about pH 7.4. However, the coexistence of other components in the dialysate, such as molecules with amine groups and carboxylic groups may outcompete urea in the binding events.

### Oxidation Based Urea Removal

Referring to FIG. 4C, another embodiment of a toxin-removal element 218 is a oxidation based element. A oxidation based urea removal element may include the oxidation cell 430 and a activated carbon cartridge 432. An advantage of urea oxidation is that electrodes have an effectively infinite storage capacity and potentially thousands of hours of operational lifetime depending on the type of electrode used. A disadvantage of the electrochemical approach can be the oxidation of the highly prevalent (about 150 mM NaCl) chloride to highly toxic chlorine. If urea-selective electrodes are developed and operational parameters are optimized, only a small amount of scavengers would be required to reduce free Cl₂ and total chlorine concentrations to the safety level compliant with FDA regulation on water quality used for dialysis.

Under certain conditions, when using platinum and other dimensionally stable anodes, urea can be oxidized into CO₂ and N₂, largely eliminating the need of sorbents and ion exchange materials for removing the partial oxidation product NH₃. However, Cl₂ and oxo-chloro species can be a by-product during urea oxidation which in many cases can be removed effectively by activated carbon. The applied bias, current density and pH all influence the efficiency and selectivity towards urea oxidation. The problems of Ca²⁺ and Mg²⁺ induced precipitates on the cathode could be eliminated by periodically switching the polarity of the anode and cathode.

Nickel oxyhydroxide (NiOOH) is one of the most urea-selective electrodes. It has urea active surface sites similar to the Ni(II) sites in urease, giving a high degree of chemical selectivity to urea over Cl⁻. However the optimal efficiency is at pH greater than 10, which is not compatible with dialysate contact with blood. Also there can be fast Ni corrosion at physiological pH decreasing performance and be potentially toxic. Ni can be doped with other metals and have nanostructured synthesis for higher performance in urea oxidation.

TiO₂ is a photooxidation catalyst that can oxidize a wide range of organics including uremic toxins. Photoelectrochemical cells with TiO₂ anode and Pt/C cathodes have shown complete oxidation to CO₂, H₂O and N₂ and a high selectivity towards urea over chloride (>90%), producing less oxidative species that need to be scrubbed with activated carbon. A photo-oxidation urea removal (POUR) element to remove urea can use hydrothermally prepared, randomly oriented, single crystalline TiO₂ nanowire meshes coated onto fluorinated tin oxide (FTO) conductive substrates.

Any organic solutes that could be oxidized, either directly on the anode or by the generated Cl₂, can produce a wide variety of by-products. Amino acids and proteins are major targets for oxidation due to their abundance and higher rate constants for reaction. Chlorination of amino acids can produce a variety of organic monochloramines or organic dichloramines, depending on the chlorine to amino acid ratio. The organic chloramines could further degrade into aldehydes, nitriles and N-chloraldimines (chlorination of amino acids). Tryptophan for example, has phenol and amino acid as the reactive functional group. With Cl:AA=0.8, N-monochlorotyrosine was formed. At Cl:AA=2.8, chlorine substitution on the aromatic ring was observed in NMR, and a mixture of N-monochloro-3-chlorotyrosine and N,N-dichlorotyrosine in a slightly lower than 1:1 ratio was formed. Thus for oxidation strategies, it is beneficial to reduce the amount of chemical species to process in order to reduce the amount of oxidation by-products.

In selecting the toxin-removal element 218 in FIG. 2, a consideration of the specific application and advantages of each will need to be considered. Enzymatic systems have high selectivity but complexity of NH₄⁺ capture and stability issues. Adsorption systems are simple to produce and operate but the binding capacity at physiological conditions might be insufficient, and can be impractical based on size and weight requirements. Oxidation systems are durable and would not need repeated replacement, however low urea selectivity can result in oxidation of nutrients and production of oxidative byproducts.

An advantage of urease based dialysis devices is the specific targeting of urea. On the other hand, the requirements for ammonia removal using zirconium-based sorbents puts constraints on the whole system. It has been estimated that 100-500 g of sorbent cartridge would last 12-24 h. Due to the removal of non-sodium cations, extra electrolyte infusates are needed to maintain the electrolyte balance. Furthermore, to ensure adequate ammonium removal in the recycled dialysate, extra zirconium phosphate has to be used to guarantee the safety margin, and blood ammonium concentration has to be closely monitored.

### Toxin-Selective Membranes

The present disclosure aims to provide a toxin-selective membrane 212 that is highly permeable to urea but can block other compounds that can interfere with the toxin-removal element 218 from passing from the dialysate loop 204 to the toxin-removal loop 206.

Generally, dialysis membranes focus on pore size while sharpening the molecular weight cutoff of high-flux membranes to maximize removal of low molecular weight proteins. Each membrane has a specific area and profile that is dependent on other variables, such as thickness, ζ-potential, molecular weight retention onset (MWRO), molecular weight cutoff (MWCO), etc.

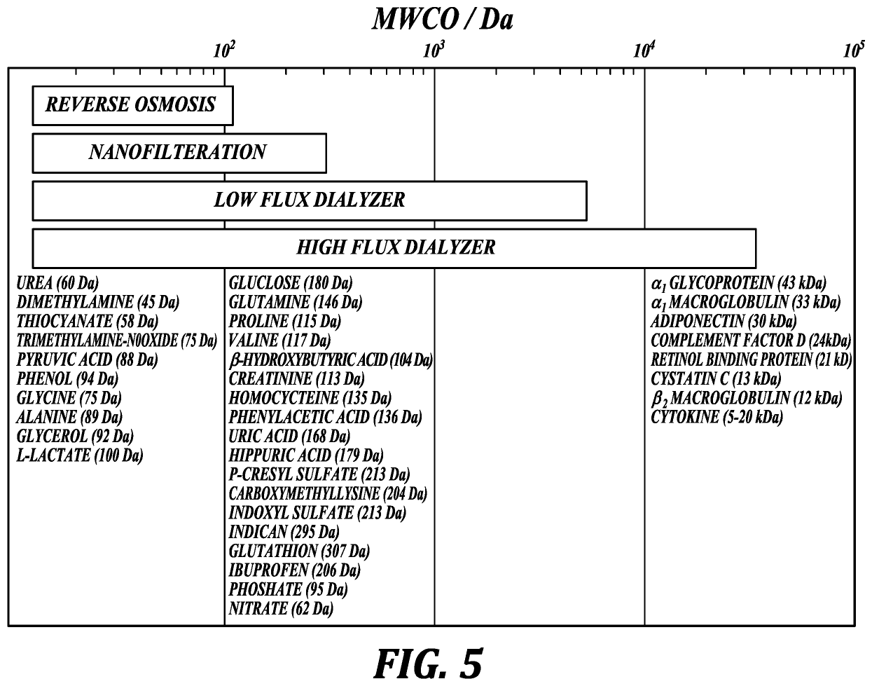

FIG. 5 shows the MWCO of four classes of dialysis membranes 210. FIG. 5 lists the prevalent toxins and nutrients. Low flux dialyzers have MWCO of about 5 kDa while high flux dialyzers are near about 20 kDa to target O₂-macroglobulin. Nanofiltration membranes can have MWCO nominally near 100 Da, but 200 Da is more common in practice.

Reverse osmosis (RO) membranes are a class of membranes with sub-nanometer channels that are highly effective on excluding charged species even at low MW, such as Na⁺ at 23 Da, so a formal MWCO does not generally apply. Neutral small organics such as urea have high permeability through RO membranes. The list shows that there is no MWCO that will allow only uremic toxins to pass but not nutrients such as glucose (180 D) and amino acids (75-204 D).

Table 1 below lists the 20 prevalent small molecules (MW<200 Da) removed in thrice weekly hemodialysis session.

| | Molecular | pre-HD | Total mass |
|---|---|---|---|
| | weight | concentration | removal |
| Molecule | (Da) | (mg/L) | (g) |
| Glucose | 180 | 972 | 24.0 |
| Urea | 60 | 1260 | 23.6 |
| L-lactate | 100 | 150 | 3.7 |
| Phenylacetic acid | 136 | 456 | 2.2 |
| Creatinine | 113 | 99.0 | 1.65 |
| Glutamine | 146 | 38.1 | 1.44 |
| Uric acid | 168 | 78.8 | 1.01 |
| Glycerol | 92 | 40.5 | 0.99 |
| Phenylacetylglutamine | 264 | 4.43 | 0.82 |
| Proline | 115 | 32.7 | 0.718 |
| Valine | 117 | 23.5 | 0.505 |
| Glycine | 75 | 39.4 | 0.477 |
| Glutamate | 147 | 19.7 | 0.476 |
| Alanine | 89 | 20.5 | 0.470 |
| Hippuric acid, total | 179 | 42.7 | 0.464 |
| Leucine | 131 | 16.4 | 0.424 |
| Lysine | 131 | 20.3 | 0.424 |
| Arginine | 172 | 17.7 | 0.372 |
| Phenylalanine | 165 | 11.2 | 0.277 |
| Isoleucine | 131 | 20.3 | 0.252 |
| Total (top 20) | | | 64.3 |
| Total (top 50) | | | 67.6 |

The total mass removal of expected species of molecular weight less than 200 Da (achievable through commercially available nanofiltration membranes) is over 64.3 g (or 67.6 g for the top 50 most removed small molecules). Of this total, about 23.6 g is the target urea with a remainder 44.0 g of non-urea small molecules (186% increase in mass). For non-selective, oxidation-based urea removal/degradation systems, a proportionately larger system would be required. The complete oxidation of urea, with 60 Da MW, is a 6 e⁻ process. Considering an average body composition (C:H:N:O ratio) gives an 8.1 e−/60 Da, this would further increase the current requirement 35% compared to urea alone. The excess mass and current requirements would require the oxidation unit to have a about 251% increase in capacity, or almost triple in size due to saturating the system with competing chemistries.

By using, for example, a 100 Da MWCO membrane, such as NF90 (nominal 90 Da MWCO) instead the mass of non-urea small molecules (from Table 1) would be reduced from 44.0 g to 9.3 g (with MW<120), dramatically reducing saturation by competition. The mass of non-urea small molecules with MW<120 should be <1 g. Accounting for the increase in current required for non-urea oxidation (about 8.1 e⁻), the unit would have to be only 53% larger if a 100 Da MWCO membrane is used (or <6% increase).

Accordingly, oxidation by-products can be numerous unless a toxin-selective membrane 212 and toxin-removal loop 206 is used before an oxidation-based urea removal element 218.

The toxin-removal loop 206 and toxin-selective membrane 212 can also aid urease-based urea removal/degradation systems, by blocking interfering species, such as amino acids. The urease is fixed onto the cartridge by either physical adsorption or click chemistry, but, there can be leaching of urease due to competition of binding with scaffold materials from amino acids. This urease release could produce toxic ammonia concentrations downstream from the ammonia adsorption system and contact the blood dialyzer. The toxin-removal loop 206 can include ammonia removing sorbents that allows any residue ammonia to circulate through the ammonia removing sorbents. A reduction of amino acids by the toxin-removal loop 206 also increases enzyme activity by preventing interference with enzyme active sites.

Similarly, the sorbent-based urea removal would also benefit from the toxin-removal loop 206 and toxin-selective membrane 212 by restricting the amounts of competing solutes. Sorbent binding mechanisms typically depend on the interactions with amine group of the urea molecule. However, the combined concentration of amino acids in healthy individuals are already greater than 3 mM, not counting polypeptides and protein molecules. This is the likely mechanism for the precipitous drop in urea binding capacities for potential sorbents when they were switched from PBS buffer to real spent dialysate solutions.

The toxin-removal loop 206 and a toxin-selective membrane 212, for example, a urea-selective membrane can benefit the above-mentioned modes of urea removal, enhancing the safety and efficiency while reducing the cost. The urea-selective membrane 212 will also benefit the portablization of a hemodiafiltration device, where the same urea removal strategies still apply, and non-urea uremic toxin removal could be enhanced comparing to hemodialysis. In the case of oxidative urea removal approaches, there may be no need for the specialty zirconium phosphate or zirconium oxide ion exchange materials, only activated carbon which is a very inexpensive consumable.

Implementing a toxin-removal loop in wearable and portable kidney dialysis devices may place new requirements on the membrane at the blood interface, where the sieving coefficient can be a useful parameter characterizing the potential of solutes (most importantly, middle molecules) to pass across the dialyzer membrane 210, particularly under conditions of convective flow with patient fluid level management. At the interface between the dialysate loop 204 and the toxin-removal loop 206, the membrane 212 should have a high selectivity towards urea versus other small molecules, to further enhance functionality of the urea-removal element 218.

In one embodiment, the urea-selective membrane 212 could be based on size exclusion using membranes that are shrunk controllably from larger pores. In one embodiment, cellulose acetate membranes may be used as the toxin-removal membrane 212 for separating urea. In one embodiment, the toxin-removal membrane 212 can be an commercially available polymer based, polyamide based, or other polymeric based membranes used in reverse osmosis (RO), low pressure RO, forward osmosis (FO), and nanofiltration (NF). A forward osmosis (FO) membrane allows the loop to operate without mechanical pressure and balance liquid levels. Transport operates by diffusion across the FO membrane. A reverse osmosis (RO) membrane requires mechanical pressure; rejects salts (Cl—); toxin transport is a combination of diffusion and fluid flow; and provides no liquid balancing.

For pressure driven flow through RO and NF membranes, urea has a low rejection rate of 18% and 22% respectively. In contrast, creatinine has a high rejection rate that only dropped slightly from 96% to 89% when switched from RO to NF membrane. In the RO case, the urea/creatinine selectivity is 20:1 and NF is 7:1. Higher selectivity can be expected for most of the prevalent blood serum molecules and this 20:1 selectivity factor is more than required to protect oxidation units from saturation. The easy flow of urea through commercial RO membranes has been a long-standing problem in wastewater treatment, while in present application, it can be beneficial to have selectivity for urea transport.

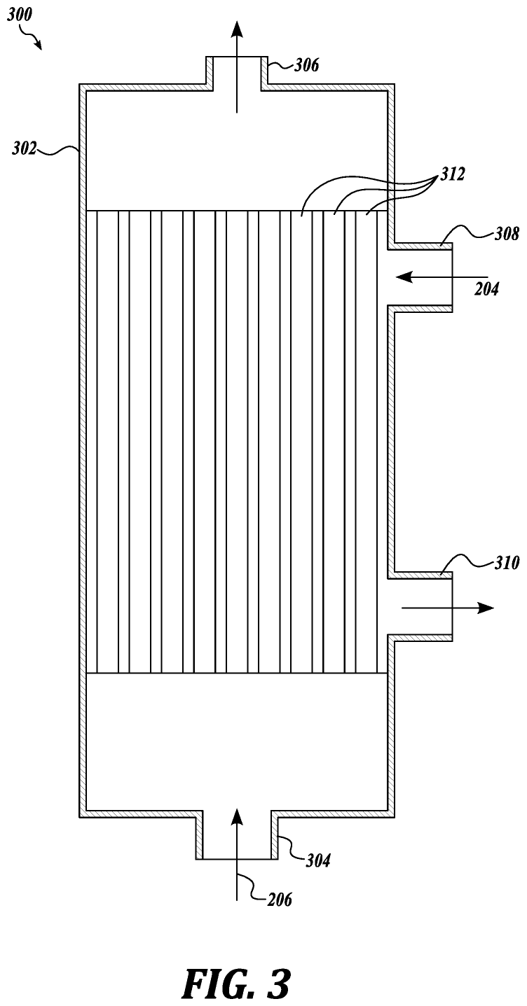

In one embodiment, a commercially available, hollow fiber FO membrane is used for the toxin-selective membrane 212 in the toxin-removal loop 206. FIG. 3 is a schematic illustration of a device 300 including a plurality of hollow fiber membranes 312. The device 300 can be incorporated into the circuit 200 of FIG. 2 for use as the toxin-selective membrane 212 for separating the dialysate loop 204 from the toxin-removal loop 206.

Generally, a hollow fiber membrane device 300 includes a plurality of hollow fibers 312 formed having a permeable membrane separating the interior from the exterior of the hollow fibers. Accordingly, the device 300 is configured to separate a first fluid flowing within the hollow fibers 312 from a second fluid flowing over the exterior of the hollow fibers 312. For example, the hollow fibers 312 can be arranged so that each end of each of the hollow fibers 312 passes through and terminates at a bulkhead, thereby sealing the interior channel of the hollow fibers 312 from the exterior of the hollow fibers. This allows flow of a first fluid within the hollow fibers 312 in the axial direction, and a second fluid to flow over the exterior of the hollow fibers 312 to cause the migration of chemical species across the hollow fibers 312. For example, the toxin-removal loop 206 flow enters nozzle 304 and exits through nozzle 306 thereby passing within the inside of the hollow fibers 312. The dialysate loop 204 flow can enter through either of nozzles 308 or 310 and exit through the other. The toxin, such as urea, can migrant from the dialysate loop 204 to the toxin-removal loop 206 through the membrane forming the walls of the hollow fibers 312.

In one embodiment, the toxin-removal loop 206 includes a photoelectrochemical urea oxidation system as the urea-removal element 218. In one embodiment, the hollow fiber membrane 212 is operated in forward osmosis mode. In one embodiment, a hollow fiber membrane operated in forward osmosis mode can transport about 15 g/day to 30 g/day of urea at about 10 mM concentration gradient and an inner fiber flow rate of 50 mL/min. About 30 g/day is more than twice the daily urea generation rate of a patient and corresponds to a mass transfer coefficient of 1.2 μm/s. In one embodiment, fluid transfer rate across the hollow fiber membrane was sufficient for the removal of up to 4.5 L of water by evaporation in the oxidation system. Photo-electrochemical oxidation performance was enhanced threefold using the hollow fiber membrane as the toxin-selective membrane 212 in a toxin-removal loop 206, and was shown to be safe by cytotoxicity studies.

In one embodiment, an activated carbon cartridge 214 is used in the dialysate loop 204 of FIG. 2 to remove non-urea uremic toxins. In one embodiment, a liquid dialysis circuit can include the dialysate loop 204 and a toxin-removal loop 206 separated by a toxin-selective membrane 212 configured to selectively pass a toxin from the dialysate loop to the toxin-removal loop. In one embodiment, the toxin-selective membrane 212 includes hollow fiber membranes. In one embodiment, the toxin-selective membrane has a surface area for mass transfer of at least 2.4 m². In one embodiment, the toxin-selective membrane has a surface area for mass transfer from 1 to 5 m². In one embodiment, the toxin-selective membrane has a surface area for mass transfer from 2 to 4 m². In one embodiment, the toxin-removal loop flows inside of the hollow fiber membranes, and the dialysate loop flows on the outside of the hollow fiber membranes. In one embodiment, the flow of the toxin-removal loop is at least 36 mL/min. In one embodiment, the flow of the toxin-removal loop is from 10 to 100 mL/min. In one embodiment, the flow of the toxin-removal loop is from 10 to 80 mL/min. In one embodiment, the flow of the toxin-removal loop is from 20 to 50 mL/min. In one embodiment, the flow of the dialysate loop is at least 3.7 times the flow of the toxin-removal loop. In one embodiment, the flow of the dialysate loop is 1 to 5 times the flow of the toxin-removal loop. In one embodiment, the flow of the dialysate loop is 2 to 4 times the flow of the toxin-removal loop. In one embodiment, the dialysate loop includes a uremic toxin.

In view of the foregoing, representative embodiments can include, but are not limited to the following.

1. A liquid dialysis circuit, comprising a dialysate loop 204, separated from a patient blood circuit 202 by a dialysis membrane 210, and a toxin-removal loop 206 separated by a toxin-selective membrane 212 configured to selectively pass the toxin from the dialysate loop 204 to the toxin-removal loop 206.

2. The liquid dialysis circuit of Embodiment 1, wherein the toxin is a uremic toxin.

Representative uremic toxins include urea, creatinine, indoxyl sulfate, uric acid, and phenylacetic acid.

3. The liquid dialysis circuit of Embodiment 1, wherein the toxin-selective membrane 212 is an osmotic membrane.

4. The liquid dialysis circuit of Embodiment 1, wherein the toxin-selective membrane 212 is configured to operate as a forward-osmosis membrane.

A forward osmosis (FO) membrane allows the circuit to operate without mechanical pressure and balance liquid levels. Transport operates by diffusion across the FO membrane.

5. The liquid dialysis circuit of Embodiment 1, wherein the toxin-selective membrane 212 is configured to operate as a reverse-osmosis membrane.

A reverse osmosis (RO) membrane requires mechanical pressure; rejects salts (Cl—); toxin transport is a combination of diffusion and fluid flow; and provides no liquid balancing.

6. The liquid dialysis circuit of any of the preceding Embodiments, wherein the toxin-selective membrane 212 operates based on the mechanism of ion-exchange (to pursue negative-charged toxins), affinity binding, ultrafiltration, reverse osmosis or forward osmosis.

7. The liquid dialysis circuit of any of the preceding Embodiments, wherein the toxin-removal loop 206 comprises non-biocompatible liquid.

Such an embodiment allows for operation without NaCl solution. Examples include NaHCO₃ and NaOH. The loop can also operate at non-physiological pH. “Non-biocompatible” includes chemistry and pH, CO2 level, ammonia, total chlorine (Cl gas, ions, and Cl-containing species—oxidative), and toxic metal ions.

8. The liquid dialysis circuit of any of the preceding Embodiments, wherein the dialysate loop 204 comprises a liquid filter 214 configured to remove a uremic toxin from a dialysate liquid flowing through the dialysate loop 204.

Activated charcoal is a typical filter material, but any other compatible filter or method can also be used.

9. The liquid dialysis circuit of any of the preceding Embodiments, wherein the toxin-removal loop 206 comprises a liquid filter 216 configured to remove a uremic toxin or oxidation product from a toxin-removal liquid flowing through the toxin-removal loop 206.

10. The liquid dialysis circuit of any of the preceding Embodiments, wherein the toxin-removal loop 206 comprises a toxin-removal element 218 in liquid contact with a toxin-removal liquid flowing through the toxin-removal loop 206.

Toxin-removal elements 218 include photo-oxidation, electro-oxidation, enzyme, sorbent, and a (bacteria) bioreactor.

11. The liquid dialysis circuit of Embodiment 10, wherein the toxin-removal element 218 is selected from a photo-oxidation element, an electro-oxidation element, and combinations thereof.

12. The liquid dialysis circuit of Embodiments 10 or 11, wherein the toxin-removal element 218 is selected from enzyme (urease)-based, bacteria-based (bioreactor), sorbent-based, oxidation-based, and combinations thereof.

13. The liquid dialysis circuit of any of Embodiments 10-12, wherein the toxin-removal element 218 includes a photo-oxidation element.

14. The liquid dialysis circuit of Embodiment 13, wherein the liquid dialysis circuit is capable of filtering at least 0.625 g/hour of toxin from the dialysate loop.

15. The liquid dialysis circuit of any of the preceding Embodiments, wherein the liquid dialysis circuit is capable of removing 0.5 L-4.0 L of water from the dialysate loop 204 in 24 hours.

16. The liquid dialysis circuit of any of the preceding Embodiments, wherein the liquid dialysis circuit is configured to controllably remove water from the dialysate loop 204 via a photo-oxidation element 218 in the toxin-removal loop 206.

In certain embodiments, the photo-oxidation heats the water, which escapes through non-water-tight components in the system (e.g., a carbon cloth electrode). This evaporation can be controlled and measured to determine fluid loss in the system.

17. The liquid dialysis circuit of any of the preceding Embodiments, wherein the dialysate loop 204 is configured to contain a dialysate and for the dialysate to receive uremic blood toxins from the patient's blood across the dialysis membrane 210.

18. The liquid dialysis circuit of any of the preceding Embodiments, wherein the dialysate loop 204 is configured to received used peritoneal fluid.

19. The liquid dialysis circuit of any of the preceding Embodiments, wherein the toxin-selective membrane includes hollow fiber membranes.

20. The liquid dialysis circuit of any of the preceding Embodiments, wherein the toxin-selective membrane has a surface area for mass transfer of at least 2.4 m².

21. The liquid dialysis circuit of Embodiment 19, wherein the toxin-removal loop flows inside of the hollow fiber membranes, and the dialysate loop flows on the outside of the hollow fiber membranes.

22. The liquid dialysis circuit of Embodiment 21, wherein the flow of the toxin-removal loop is at least 36 mL/min.

23. The liquid dialysis circuit of Embodiment 22, wherein the flow of the dialysate loop is at least 3.7 times the flow of the toxin removal loop.

24. A kidney dialysis system comprising a liquid dialysis circuit of any of the preceding Embodiments.

25. The kidney dialysis system of Embodiment 24, wherein the kidney dialysis system is a hemodialysis system, a peritoneal dialysis system, a hemofiltration system, or a hemodiafiltration system.

26. The kidney dialysis system of Embodiment 24 or 25, wherein the kidney dialysis system has a form factor selected from the group of portable, wearable, movable, and fixed.

27. The kidney dialysis system of any of Embodiments 24-26, wherein the kidney dialysis system is configured for in-home or in-dialysis center use.

28. A method of treating a patient with toxified blood, comprising:
a. flowing toxified liquid from the patient into a liquid dialysis circuit of any of the preceding Embodiments, wherein the toxified liquid flows along a dialysis membrane, allowing uremic toxins to pass into the dialysate loop to produce toxified dialysate;
b. in the dialysate loop, flowing the toxified dialysate along the toxin-selective membrane to produce detoxified dialysate in the dialysate loop and toxified fluid containing electrolytes in the toxin-removal loop, wherein the detoxified dialysate is returned towards the dialysis membrane; and
c. in the toxin-removal loop, flowing the toxified fluid containing electrolytes into a toxin-removal element, to produce detoxified fluid containing electrolytes, wherein the detoxified fluid containing electrolytes is returned towards the toxin-selective membrane.

29. The method of Embodiment 28, wherein the toxified liquid is blood or peritoneal dialysis fluid.

30. The method of Embodiment 28 or 29, further comprising a step of adding electrolytes to the fluid containing electrolytes.

31. A method of regenerating a dialysate fluid, comprising:
a. flowing a toxified dialysate along a toxin-selective membrane 212 to produce detoxified dialysate in a dialysate loop 204 and toxified fluid containing electrolytes in a toxin-removal loop 206; and
b. in the toxin-removal loop 206, flowing the toxified fluid containing electrolytes into a toxin-removal element 218, to produce detoxified fluid containing electrolytes, wherein the detoxified fluid containing electrolytes is returned towards the toxin-selective membrane 212.

32. A liquid dialysis circuit, comprising:
a dialysate loop 204; and
a toxin-removal loop 206 separated by a toxin-selective membrane 212 configured to selectively pass a toxin from the dialysate loop to the toxin-removal loop.

33. The liquid dialysis circuit of Embodiment 32, wherein the toxin-selective membrane 212 includes hollow fiber membranes 312.

34. The liquid dialysis circuit of Embodiment 32 or 33, wherein the toxin-selective membrane 212 has a surface area for mass transfer of at least 2.4 m².

35. The liquid dialysis circuit of Embodiment 34, wherein the toxin-removal loop 206 flows inside of the hollow fiber membranes 312, and the dialysate loop 204 flows on the outside of the hollow fiber membranes 312.

36. The liquid dialysis circuit of Embodiment 35, wherein the flow of the toxin-removal loop 206 is at least 36 mL/min.

37. The liquid dialysis circuit of Embodiment 36, wherein the flow of the dialysate loop 204 is at least 3.7 times the flow of the toxin removal loop 206.

38. The liquid dialysis circuit of Embodiment 32, wherein the dialysate loop includes a uremic toxin.

### EXAMPLES

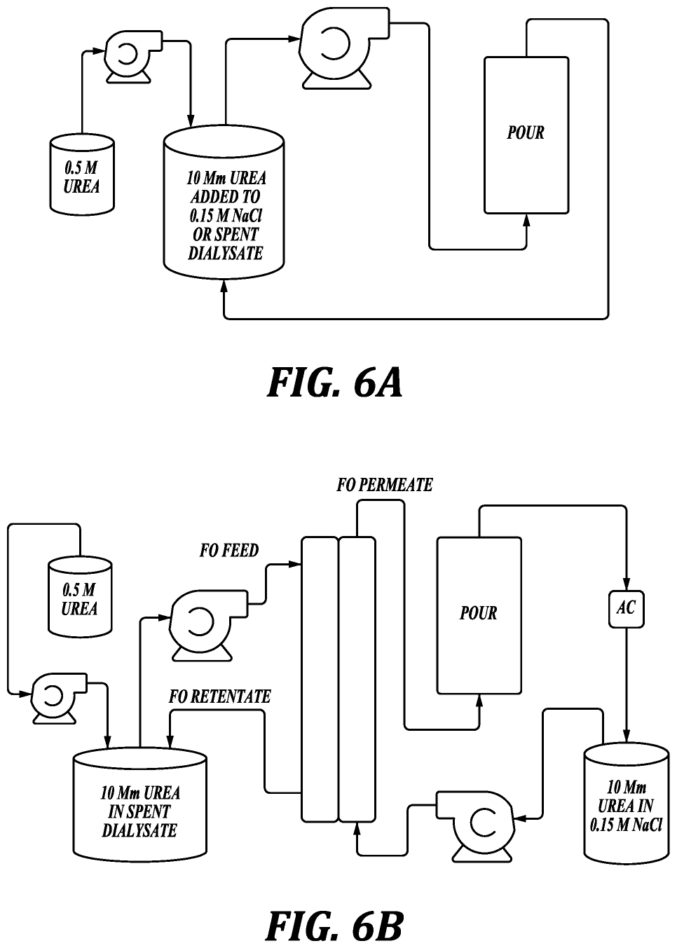

To demonstrate the impact from the other molecular species on urea, a control study of urea removal in human spent dialysate and saline solution was conducted. As shown in FIG. 6A, a previously developed photo-oxidation urea removal (POUR) device was used to remove urea. With urea infusion, both the spent dialysate and saline solutions had the same initial urea concentration (˜10 mM) at the start of the experiment, and then were continuously infused with the amount of urea that would correspond to 15 g urea/day if scaled up to ˜2000 cm², an acceptable size for a kidney dialysis system. If urea oxidation rate meets the target, the urea concentration would remain constant emulating continuous operation, analogous to the normal functioning of the kidneys. The results are shown in Table 2 below.

TABLE 2
POUR urea removal rates in the medium of 0.15M NaCl vs. human spent
dialysate with initial urea loading and 15 g/day equivalent urea infusion.

| | | Urea | Urea | Glucose | Glucose |
|---|---|---|---|---|---|
| | Time | concentration | decomposition rate | concentration | decomposition rate |
| Solution | (h) | (mM) | (mg/(cm² · h)) | (mM) | (mg/(cm² · h)) |
| 0.15M NaCl | 0 | 9.8 | 0.29 | N/A | N/A |
| | 24 | 10.6 | | N/A | |
| Human spent | 0 | 11.6 | 0.090 | 2.55 | 0.087 |
| dialysate | 24 | 20.7 | | 1.28 | |

Using a simple saline solution spiked and infused with urea, a urea removal rate was achieved of 0.29 mg/(cm²·h) which is close to the target rate of 0.31 mg/(cm²·h). This target flux corresponds to 15 g/day for our benchtop demonstration device with 2000 cm² of catalyst surface area. However, when using human spent dialysate, the urea removal rate dropped to 0.09 mg/(cm²·h), over a 3-fold drop in performance which is close to expectations for competitive oxidation (34%). The glucose removal rate was similar to urea at 0.087 mg/(cm²·h). As expected, the photocurrent that would otherwise be utilized in urea removal is now diverted to the oxidation of glucose and other organic species that were present in the human spent dialysate. Although solute interference for this photooxidation-based urea removal device was demonstrated, the interference is expected to be universal for the other oxidation-based urea removal approaches.

Due to competition from other solutes, a urea-selective membrane was used with sufficient permeability for the 15 g urea/day target. For wastewater treatment and desalination, reverse osmosis (RO) membranes have been highly effective in removing most solutes based on the principle of size and charge exclusion. However, urea is an important exception, showing only about 20% rejection in the permeate where other species are typically near 95-99% rejection. Though problematic for wastewater treatment, this shows RO membranes are highly selective to urea transport and can be used for dialysate regeneration by urea removal. To explore the selectivity of RO membrane towards the molecular species in spent dialysate, a list was compiled of 11 representative molecules including urea, glucose, amino acids and uremic toxins, as shown in Table 3 below.

TABLE 3
Prevalent molecular species in human spent dialysate and uremic blood plasma concentrations
used as feed solution for RO and FO separations. Observed RO and FO retentate concentrations
and calculated rejection ratios, removal rate and mass transfer coefficient. The flow rates used are
100 and 400 mL/min for the inner fiber and outer volume, respectively.

| | | | | | | | FO | FO mass |
|---|---|---|---|---|---|---|---|---|
| | | Feed | RO | RO | FO | FO | Removal | transfer |
| | | concentration | permeate | Rejection | retentate | permeate | Rate | coefficient |
| | MW | (mg/L) | (mg/L) | Ratio | (mg/L) | (mg/L) | (g/day) | (μm/s) |
| Urea | 60 | 600 | 465 | 22.5% | 474 | 414 | 86.4 | 0.909 |
| Glucose | 180 | 972 | 10 | 99.0% | 952 | 1 | 0.14 | 0.00075 |
| L-lactate | 100 | 150 | 10 | 93.3% | 150 | ND | ND | N/A |
| Serine | 105 | 7.2 | 0.1 | 98.6% | 6.9 | ND | ND | N/A |
| Alanine | 89 | 22.5 | 0.4 | 98.2% | 22.0 | TR | TR | N/A |
| Glycine | 75 | 17.6 | 0.8 | 95.5% | 16.9 | TR | TR | N/A |
| Tryptophan | 204 | 5.5 | ND | ~100% | 4.5 | ND | ND | N/A |
| Methionine | 146 | 3.4 | ND | ~100% | 3.3 | ND | ND | N/A |
| Glutamine | 146 | 62.5 | ND | ~100% | 58.5 | ND | ND | N/A |
| Indoxyl | 212 | 2.8 | ND | ~100% | 2.3 | ND | ND | N/A |
| sulfate | | | | | | | | |
| Creatinine | 113 | 99 | 21 | 78.8% | 94 | ND | ND | N/A |
| Phenylacetic | 136 | 100 | <5 | >95% | 96 | <5 | <0.72 | <0.038 |
| acid | | | | | | | | |
| Ammonium | 53.5 | 2.7 | 0.38 | 86.0% | 2.67 | 0.31 | 0.04 | 0.089 |
| chloride | | | | | | | | |

Urea is the most abundant uremic waste (23.6 g) while glucose is the most prevalent nutrient (19.4 g). Glycine and tryptophan are selected for their small molecular weight and characteristic functional groups, while ammonium is a potential by-product from urea photooxidation. Their respective concentrations have been reported previously. This list is highly representative of the molecular species that a urea-selective membrane will have to face in the real spent dialysate.

The results of RO selectivity are shown in Table 3. The rejection rate for urea is 20%, similar to what has been observed in wastewater treatment. The rejection rate of glucose, the most prevalent competitive solute is 99%. Amino acid nutrients range from 96-100% rejection. The feed solution had a urea:non-urea mass ratio of 1:2.4, which was decreased to 1:0.1 in the RO permeate. This effectively eliminates competitive solutes with only ˜10% expected drop in urea removal efficiency.

However, the RO mode of operation is designed to remove all solutes including NaCl, which requires about 7.5 bar of hydraulic pressure to generate the urea rich permeate. This adds significant power use and complexity, especially considering retentate and permeate streams will be remixed after the urea removal step.

As shown in FIG. 6B, the urea-selective membrane can be operated in FO mode with negligible hydraulic pressure. Water would still flow across the membrane by osmotic pressure differences in the dialysate loop and urea removal loop. This may occur due to water evaporation at the air permeable cathode of the POUR unit and thus concentrate salts in the urea removal loop, producing osmotic pressure. Urea flux would be by diffusive concentration gradient across the membrane. Diffusive flux will have to be sufficient to remove ˜15 g urea per day in practical sized membranes for portable dialysis.

Table 3 shows that 86.4 g/day urea removal is possible with the 2.3 m² hollow fiber forward osmosis (HFFO) cartridge and a 20 mM concentration gradient, far above daily requirements. The inner fiber flow path is used as the ‘draw’ solution for the urea-removal loop (output being FO permeate), while the outside fiber volume was used for the urea source of the dialysate loop (output being FO retentate). The mass transport coefficient shows urea at 0.91 μm/s, with ammonium 90% less and glucose 99.9% less, making the FO membrane highly selective to urea and a protective barrier.

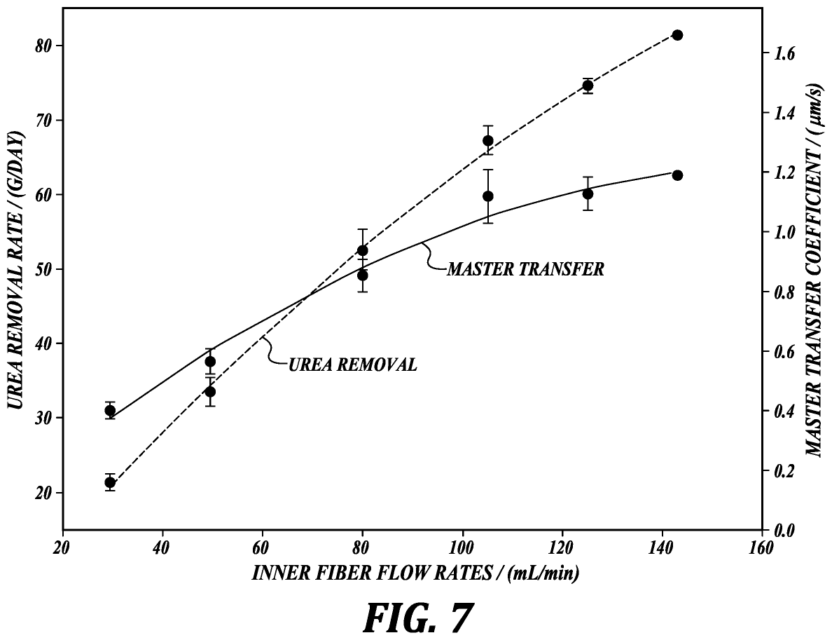

FIG. 7 shows urea removal rate in 24 h and corresponding mass transfer coefficient using the AQUAPORIN® FO cartridge at different inner fiber flow rates. Outer flow rate of 10 mM urea dialysate feed is 3.7 times the inner fiber flow rate. FIG. 7 shows the inner flow rate dependence on urea removal and mass transport coefficient with a 10 mM urea feed solution. As expected, higher flow rates will remove more urea due to the larger volumes of permeate at similar exit concentrations. With a diffusion limited process, the total flux of urea would be set by concentration drop, not flow rate, when the flow rate is fast enough to not be at saturation. This is seen by a flow independent mass transfer coefficient, occurring at inner-fiber flow rates faster than 100 ml/min with a value of 1.2 μm/s (FIG. 7). With a typical ‘skin’ layer of 100 nm for an FO membrane, this corresponds to diffusion coefficient of 1.2×10⁻⁹ cm²/s, about 4 orders of magnitude less than aqueous bulk diffusion. This is, however, sufficient for the target 15 g day using an area of 2.3 m². About 30 g urea could be removed in 24 h through the FO cartridge with a hollow fiber flow rate of 50 mL/min, assuming a constant supply of 600 mg/L (10 mM) urea on the spent dialysate side and 0 urea on the draw side (complete urea removal by the POUR unit). The FO experiment was carried out with the urea supply outside the fiber and the draw solution inside the fiber, which is opposite the conventional ‘draw’ on outside. This is because the outside volume is 5 times the inner fiber volume, thus diluting any urea flux by a factor of 5, which would make the POUR unit less efficient. Urea mass transfer rates are measured by flipping sides of the supply and draw solutions and obtained similar results of mass transfer coefficients, indicating that the asymmetric FO membrane structure does not noticeably alter urea diffusion coefficient. Water removal from the dialysate is needed for clinical applications (˜2.5 L/day) and can be achieved by higher osmotic pressure/salt concentration on the urea-removal loop. Using 1 mM NaCl draw solution against DI water, we observed about 1.25 L water removal rate per 24 h.

The urea and water removal capability of the FO-POUR device using the setup shown in FIG. 6B is demonstrated and quantified. The dialysate loop circulates 1 L of human spent dialysate collected during an in-clinic hemodialysis session. The glucose concentration was measured to be 3 mM. Urea was added to make the starting urea concentration at 10 mM and continuously infused (0.61 ml/h) with 0.5 M urea solution that would give the equivalence of 15 g/day of continuous generation of urea in ESRD patients, if the POUR unit were scaled up to 36 channels (about 2040 cm²). In the POUR loop, 0.15 M NaCl with 0.7 L volume was used as the starting solution. (Circulated at 10 mL/min.) This corresponds to a urea-removal rate of 0.32 mg/(cm²·h) which is almost the same as in saline solution without interference from other chemical species. This provides support that the FO membrane is both selective to urea transport and diffusion through the membrane in FO mode to be fast enough for clinically relevant urea removal rates.

Three separate tests of 24 h continuous operation of the FO-POUR device were conducted, with the results summarized in Table 4 below.

TABLE 4
The 24 h performance of FO-POUR device.

| | |
|---|---|
| Urea removal rate/(mg/(cm² · h)) | 0.32 (±0.01) |
| Operating current density/(mA/cm²) | 0.99 |
| Percentage of photocurrent towards urea oxidation | 91% |
| Incident LED power/(mW/cm²) | 4 |
| Water evaporation rate/(g/cm² · h) | 0.081 |
| Single channel area/cm² | 56.6 |
| Inner fiber flow rate/(mL/min) | 1.5 |
| Outer volume flow rate/(mL/min) | 10 |

Extrapolating to an active area of 2000 cm², the FO-POUR unit could remove 15.4 g urea and 3.9 L of water in 24 h meeting both clinical requirements. The extrapolated water removal of FIG. 6B was almost equally distributed from the spent dialysate loop (1.92 L) and the POUR loop (1.98 L) as expected for osmotic equilibrium with an FO membrane. The water removal rate would be determined by the evaporation through the carbon paper air permeable cathode of the POUR device. This evaporation increases the osmotic pressure in the POUR circuit solution, which will draw more water across the FO membrane. The net result is the removal of excess water from the spent dialysate.

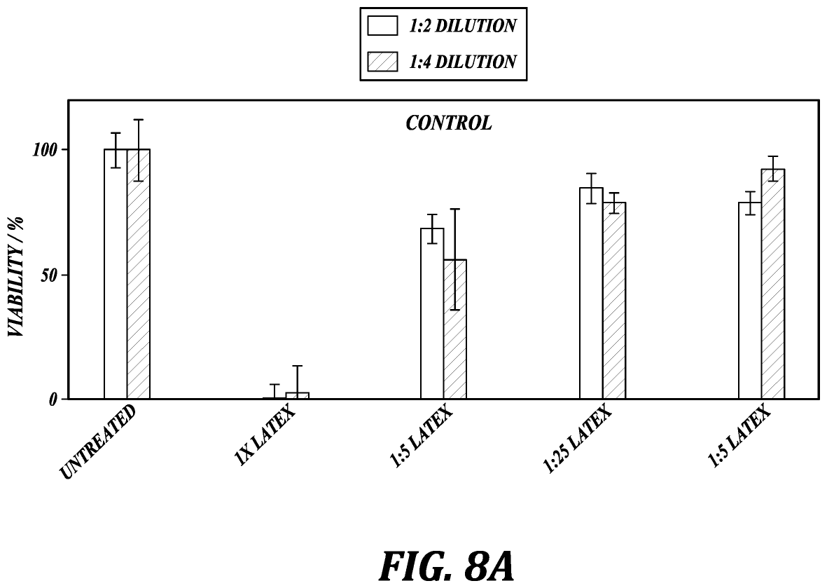

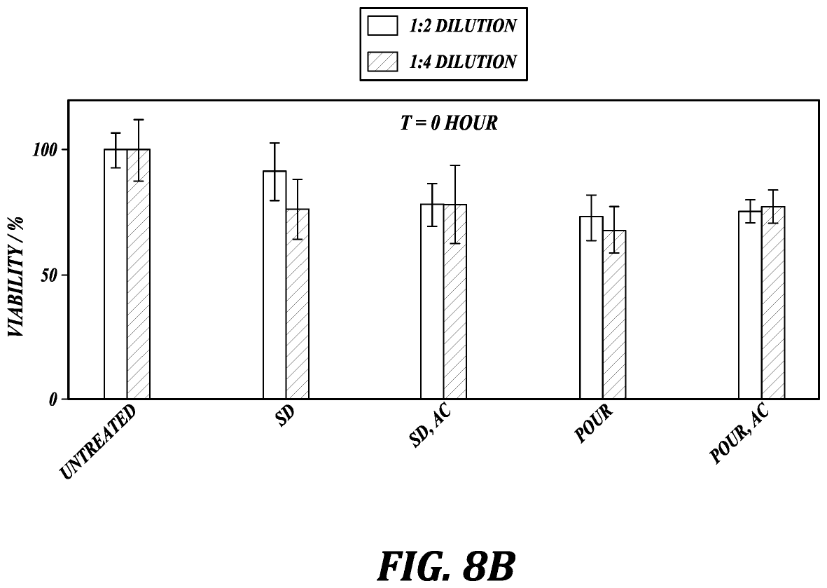

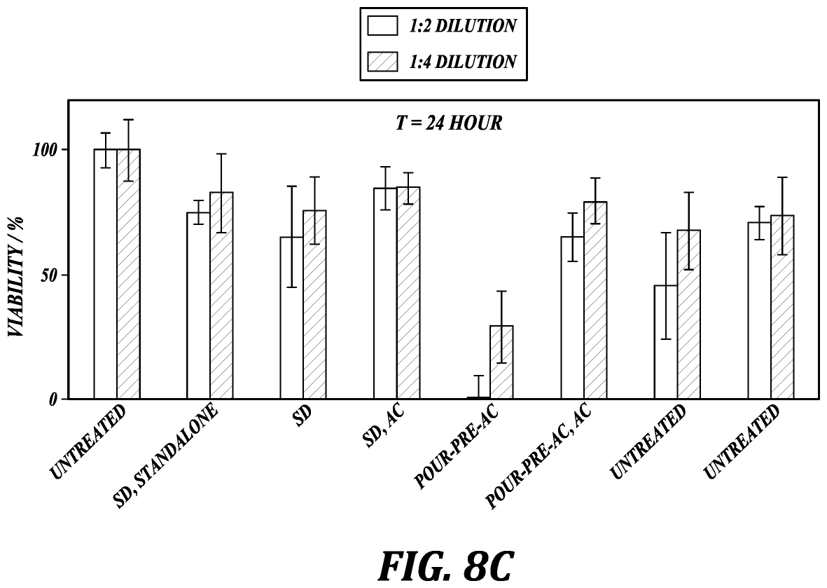

Samples were collected from both the spent dialysate and the POUR loops in the FO-POUR study (FIG. 6B) were collected, both before the after the 24 h study. The resulting MTT cytotoxicity study is summarized in FIGS. 8A, 8 B, and 8C. FIG. 8A shows the control group using the latex control diluted to different ratios. FIGS. 8B and 8C show the cytotoxicity of samples before and after the 24 h FO-POUR, respectively. At t=24 h, with a viability of 79.8% (±9.3%) of POUR output solution treated with activated carbon (AC) and 82.9% (±15.8%) from spent dialysate separate from the FO-POUR device but sitting in ambient condition for 24 h, no significant difference between their cytotoxicity were found. These viability numbers are also statistically close to the viability of 85.1% (±6.4%) of spent dialysate collected from the spent dialysate circuit at the end of the 24 h study. Activated carbon at the POUR outlet is sufficient to remove oxidative or radical species to give non-cytotoxic results. The FO membrane acts as a further protective barrier to the patient dialysate loop and does not add any cytotoxicity to the system.

Dialysate regeneration will enable portable dialysis devices that allows more frequent or near continuous treatments for better health-related outcomes for ESRD patients. However human spent dialysate is a complex solution with urea being just 40% of the target solute removal mass. This would correspond to about ⅔ drop in oxidative urea removal system performance due to competitive reactions. This was confirmed experimentally by using urea infused human spent dialysate through the POUR system giving a 69% drop in performance compared to urea infused saline solutions. A urea-selective membrane is needed to limit the exposure of oxidative systems to competing solutes and simultaneously minimize oxidation by-products from entering the regenerated dialysate and ultimately the patient body. For water purification applications, RO membranes have a characteristic of high urea transport (20% rejection). This characteristic allows these membranes to be used as a urea-selective membrane to protect urea oxidation systems from other small molecules. Rejection ratios for common uremic species were measured to be typically above 95%, except for creatinine and ammonium near 80% rejection. A useful mode of operation is in the FO configuration where urea is diffusing across the osmotic membrane without applied hydraulic pressure. Using a commercially available hollow fiber, forward osmosis cartridge, urea could be removed from the spent dialysate at a rate of over 30 g per day with high selectivity at an inner fiber flow rate of 50 mL/min and 600 mg/L (10 mM) urea concentration in the human spent dialysate feed stream. The diffusion rate of urea is 1 to 5 orders magnitude higher than other representative molecular species removed during a dialysis session. Mass transport coefficient of urea was as high as 1.2 μm/s corresponding to a diffusion coefficient of 1.2×10⁻⁹ cm²/s. With the combined FO-POUR setup, urea can be removed at a therapeutically useful rate of 0.32 mg/(cm²·h), and water removal rate of 0.081 g/(cm²·h). Additionally, there appear to be no significant differences in the cytotoxicity between untreated and treated spent dialysate. Sufficient urea removal from spent dialysate is thus achievable using an FO-POUR device, without urease enzyme and accompanying ion exchange materials, which can provide a useful portable dialysis device.

### Experimental Section

Reverse osmosis: MEMBRANE SOLUTIONS® 75 GPD RO coiled flat sheet membrane was used with an AQUATEC® 6800 Booster Pump. The flow rate was controlled with NXSTOP LZT™ M-15 0.05-0.5 GPM flow meters and calibrated using graduated cylinders. The retentate flow rate was 190 mL/min vs. permeate side flow rate of 90 mL/min, with a pressure differential of 150 psi.

Forward osmosis: AQUAPORIN INSIDE® hollow fiber forward osmosis (HFFO) membrane HFFO2-220 was used. MASTERFLEX® L/S variable speed peristaltic pumps with 77800-60 pump heads were used. The flow rates through the inner fiber and outer space were set to be between 20-160 and 80-640 mL/min, respectively. Before each sample collection, the cartridge was fully drained and then rinsed thoroughly with fresh solution 10× the volume of the FO cartridge. Inner fiber volume was 28 mL, outer volume 156 mL. Mass transfer coefficient (k) is calculated by k=\dot{n}/(AΔc). \dot{n} is the urea molar transfer rate (mol/s) obtained by the inner fiber outlet's urea concentration multiplied by the inner fiber-outlet's volumetric flow rate. A is the membrane surface area (2.3 m²). Δc is the average concentration gradient across the membrane, which is the average of fiber-inlet and outlet concentrations minus average of outer-volume inlet and outlet concentrations.

### Chemical Quantifications:

Urea, creatinine, glucose, lactose and ammonium ion are quantified following the Beckman Coulter AU System using corresponding kits with standard operating protocols. Calibration curves for urea concentration measurements were prepared using standard solutions with urea concentrations of 2, 4, 6, 8, 10 mM urea with quadratic fitting curves.

Indoxyl sulfate was quantified using liquid chromatography-tandem mass spectroscopy following a procedure described in literature. Indoxyl sulfate was first extracted by solid phase extraction and then reconstituted in 80 μL of 5% acetonitrile/0.2% formic acid in water. The sample solution was further filtered and a volume of 5 μL was injected onto a RESTEK® PFPP chromatographic column (Restek PN: 9419252) and developed using a gradient (SHIMADZU® NEXERA® XR 20A). The eluate from column was introduced into a triple quadrupole tandem mass spectrometer (SCIEX® 6500). 5 replicates were used to obtain to an average peak area.

Amino acids were quantified using standard clinical protocols. Samples were combined with internal standard (S-(2-aminoethyl)-L-cysteine, SIGMA-ALDRICH® catalog #A2636) dissolved in SERAPREP™ (Pickering Laboratories, catalog #SP100), vortexed and centrifuged to remove any precipitate. Supernatant was injected onto a BIOCHROM® 30+ amino acid analyzer (Biochrom Ltd, Cambridge, England), an HPLC system that uses a series of lithium citrate buffers to separate free amino acids with baseline resolution. Each amino acid reacts with ninhydrin in a post-column reaction for detection by UV absorbance at 570 and 440 nm. The amount of each amino acid is calculated based on two-point standard curves made with stock standards at 250 and 500 μM, respectively. Data was analyzed and quantified using OPENLAB® (EZ CHROM™, Biochrom Ltd, Cambridge, England).

Phenylacetic acid was quantified based on the absorption peak intensity at 280 nm using MOLECULAR DEVICES® SPECTRAMAX® i3x spectrometer using 4-point calibration with 2× dilution in each step starting with 0.5 mg/L phenylacetic acid in aqueous solution.

Cytotoxicity: All reagent calibrations, elutions, and cell and assay incubations were done at 37° C., 5% CO₂. Growth media was high glucose DMEM free of phenol red with final concentrations of 10% calf serum and 2 mM L-glutamine. Log phase 3T3 mouse fibroblast cells were plated in multiwell plates to be sub-confluent at 24 hours. At the same time, latex strips were eluted in growth media (0.2 g/ml) for 24 hours and used as a positive control for cytotoxicity. Liquid samples were diluted in a mix of 2× and 1× growth media to final concentration of 1× growth media. At 24 hours, eluted latex media or diluted liquid samples were transferred to quadruplicate cell wells to incubate for 48 hours. Quadruplicate wells receiving untreated growth media were used as the negative control and baseline for calculations. Unseeded wells receiving growth media were used to calculate the background.

At 48 hours, all wells were visually evaluated and assigned an ISO 10993-5 numerical score followed by the MTT cell proliferation assay (ABCAM™ 211091). Live cells convert water soluble 3-(4,5 dimethylthiazol-2-yl)-2,5-diphenyltetrazolium bromide compound to an insoluble formazan product that serves as a colorimetric marker of viability. Media was aspirated from cell wells and replaced with MTT reagent diluted in serum-free growth media. After a 3-hour incubation, well contents were aspirated and MTT solvent was added. Plates were wrapped in foil, shaken on an orbital shaker for 15 minutes, and read at absorbance 590 nm (BIOTEK® Synergy LX plate reader). Percent viability was per sample and was the average of: [sample−background)/(negative control−background)×100].

POUR device assembly and testing: TiO₂ coated FTO/borosilicate substrates were prepared hydrothermally following previous procedures. 4 mg/cm² Pt black-coated carbon paper was purchased from the FUEL CELL STORE™ (#1610009-1). Fluorinated tin oxide (FTO) on borosilicate glass slides (2.54 cm×7.62 cm) was purchased from MTI corporation (FTO257630TEC7gp25). TiO₂-coated FTO, Pt black coated carbon paper and laser cut acrylic frames (⅛″ thick clear acrylic frames 85635K451 purchased from MCMASTER-CARR®) were assembled using LOCTITE® EA M-21HP epoxy. The flow channel thickness is 3 mm and the width is 23 mm. 3 slides are used in series to give a channel length of 24.6 cm. Adapters used are COLE PARMER® MASTERFLEX® fitting, female luer to hose barb, ¼″ ID (EW-45502-20) and male luer×¼-28 UNF (EW-45505-82). SAINT GOBAIN® TYGON® 3350 ¼*⅜ silicone tubing was used to make connections between adapters. LED light panels were assembled from realUV™ LED strip lights from WAVEFORM LIGHTING™ on aluminum sheets (McMaster 89015K38, cut to 5″×10″, 360 LEDs per panel). The LED strips on each panel were soldered into two parallel circuits. The exposed edges of aluminum sheets were protected using KAPTON® polyimide film (3M® Tape 5413). FILMGRADE™ DC power supply was used to power the LED panels. METROHM® Autolab PGSTAT204 was used to apply bias and record photocurrent.

Pumps: ISMATEC® peristaltic pump 78017-10 was used at a flow rate of 1.5 mL/min for the interference experiments shown in FIG. 6A. COLE PARMER® MASTERFLEX® L/S model 77200-62 pumps were used for RO/FO experiments to obtain data in Table 3 at flow rates of 100 mL/min and 400 mL/min through the inside and outside of the hollow fiber, respectively. For the 24 h experiments, to confirm urea removal efficiency in spent dialysate, An ISMATEC® pump was used at 1.5 mL/min for the POUR side circulation and a COLE PARMER® MASTERFLEX® pump was used at a flow rate of 10 mL/min for the circulation of spent dialysate.

While illustrative embodiments have been illustrated and described, it will be appreciated that various changes can be made therein without departing from the spirit and scope of the invention.

The embodiments of the invention in which an exclusive property or privilege is claimed are defined as follows:

1. A liquid dialysis circuit, comprising a dialysate loop, separated from a patient blood circuit by a dialysis membrane, and a toxin-removal loop separated by a toxin-selective membrane configured to selectively pass the toxin from the dialysate loop to the toxin-removal loop.

2. The liquid dialysis circuit of claim 1, wherein the toxin is a uremic toxin.

3. The liquid dialysis circuit of claim 1, wherein the toxin-selective membrane is an osmotic membrane.

4. The liquid dialysis circuit of claim 1, wherein the toxin-selective membrane is configured to operate as a forward-osmosis membrane.

5. The liquid dialysis circuit of claim 1, wherein the toxin-selective membrane is configured to operate as a reverse-osmosis membrane.

6. The liquid dialysis circuit of any of the preceding claims, wherein the toxin-selective membrane operates based on the mechanism of ion-exchange, affinity binding, ultrafiltration, reverse osmosis or forward osmosis.

7. The liquid dialysis circuit of any of the preceding claims, wherein the toxin-removal loop comprises non-biocompatible liquid.

8. The liquid dialysis circuit of any of the preceding claims, wherein the dialysate loop comprises a liquid filter configured to remove a uremic toxin from a dialysate liquid flowing through the dialysate loop.

9. The liquid dialysis circuit of any of the preceding claims, wherein the toxin-removal loop comprises a liquid filter configured to remove a uremic toxin or oxidation product from a toxin-removal liquid flowing through the toxin-removal loop.

10. The liquid dialysis circuit of any of the preceding claims, wherein the toxin-removal loop comprises a toxin-removal element in liquid contact with a toxin-removal liquid flowing through the toxin-removal loop.

11. The liquid dialysis circuit of claim 10, wherein the toxin-removal element is selected from a photo-oxidation element, an electro-oxidation element, and combinations thereof.

12. The liquid dialysis circuit of claim 10 or 11, wherein the toxin-removal element is selected from enzyme (urease)-based, bacteria-based (bioreactor), sorbent-based, oxidation-based, and combinations thereof.

13. The liquid dialysis circuit of any of claims 10-12, wherein the toxin-removal element includes a photo-oxidation element.

14. The liquid dialysis circuit of claim 13, wherein the liquid dialysis circuit is capable of filtering at least 0.625 g/hour of toxin from the dialysate loop.

15. The liquid dialysis circuit of any of the preceding claims, wherein the liquid dialysis circuit is capable of removing 0.5 L-4.0 L of water from the dialysate loop in 24 hours.

16. The liquid dialysis circuit of any of the preceding claims, wherein the liquid dialysis circuit is configured to controllably remove water from the dialysate loop via a photo-oxidation element in the toxin-removal loop.

17. The liquid dialysis circuit of any of the preceding claims, wherein the dialysate loop is configured to contain a dialysate and for the dialysate to receive uremic blood toxins from the patient's blood across the dialysis membrane.

18. The liquid dialysis circuit of any of the preceding claims, wherein the dialysate loop is configured to received used peritoneal fluid.

19. The liquid dialysis circuit of any of the preceding claims, wherein the toxin-selective membrane includes hollow fiber membranes.

20. The liquid dialysis circuit of any of the preceding claims, wherein the toxin-selective membrane has a surface area for mass transfer of at least 2.4 m².

21. The liquid dialysis circuit of claim 19, wherein the toxin-removal loop flows inside of the hollow fiber membranes, and the dialysate loop flows on outside of the hollow fiber membranes.

22. The liquid dialysis circuit of claim 21, wherein the flow of the toxin-removal loop is at least 36 mL/min.

23. The liquid dialysis circuit of claim 22, wherein the flow of the dialysate loop is at least 3.7 times the flow of the toxin removal loop.

24. A kidney dialysis system comprising a liquid dialysis circuit of any of the preceding claims.

25. The kidney dialysis system of claim 24, wherein the kidney dialysis system is a hemodialysis system, a peritoneal dialysis system, a hemofiltration system, or a hemodiafiltration system.

26. The kidney dialysis system of claim 24 or 25, wherein the kidney dialysis system has a form factor selected from the group of portable, wearable, movable, and fixed.

27. The kidney dialysis system of any of claims 24-26, wherein the kidney dialysis system is configured for in-home or in-dialysis center use.

28. A method of treating a patient with toxified blood, comprising:
a. flowing toxified liquid from the patient into a liquid dialysis circuit of any of the preceding claims, wherein the toxified liquid flows along a dialysis membrane, allowing uremic toxins to pass into the dialysate loop to produce toxified dialysate;
b. in the dialysate loop, flowing the toxified dialysate along the toxin-selective membrane to produce detoxified dialysate in the dialysate loop and toxified fluid containing electrolytes in the toxin-removal loop, wherein the detoxified dialysate is returned towards the dialysis membrane; and
c. in the toxin-removal loop, flowing the toxified fluid containing electrolytes into a toxin-removal element, to produce detoxified fluid containing electrolytes, wherein the detoxified fluid containing electrolytes is returned towards the toxin-selective membrane.

29. The method of claim 28, wherein the toxified liquid is blood or peritoneal dialysis fluid.

30. The method of claim 28 or 29, further comprising a step of adding electrolytes to the fluid containing electrolytes.

31. A method of regenerating a dialysate fluid, comprising:
a. flowing a toxified dialysate along a toxin-selective membrane to produce detoxified dialysate in the dialysate loop and toxified fluid containing electrolytes in a toxin-removal loop; and
b. in a toxin-removal loop, flowing the toxified fluid containing electrolytes into a toxin-removal element, to produce detoxified fluid containing electrolytes, wherein the detoxified fluid containing electrolytes is returned towards the toxin-selective membrane.

32. A liquid dialysis circuit, comprising:
a dialysate loop; and
a toxin-removal loop separated by a toxin-selective membrane configured to selectively pass a toxin from the dialysate loop to the toxin-removal loop.

33. The liquid dialysis circuit of claim 32, wherein the toxin-selective membrane includes hollow fiber membranes.

34. The liquid dialysis circuit of claim 32 or 33, wherein the toxin-selective membrane has a surface area for mass transfer of at least 2.4 m².

35. The liquid dialysis circuit of claim 34, wherein the toxin-removal loop flows inside of the hollow fiber membranes, and the dialysate loop flows outside of the hollow fiber membranes.

36. The liquid dialysis circuit of claim 35, wherein the flow of the toxin-removal loop is at least 36 mL/min.

37. The liquid dialysis circuit of claim 36, wherein the flow of the dialysate loop is at least 3.7 times the flow of the toxin removal loop.

38. The liquid dialysis circuit of claim 32, wherein the dialysate loop includes a uremic toxin.
